(19) United States
(12) Plant Patent Application Publication
(10) Pub. No.: US 2019/0021197 P1
(43) Pub. Date: Jan. 17, 2019

(54) Hydrangea Plant Named 'H216906'

(50) ;

(71) Applicant: Niels Arts, Aalsmeer (NL)

(72) Inventor: Niels Arts, Aalsmeer (NL)

(73) Assignee: HYDRANGEA BREEDERS ASSOCIATION B.V., De Kwakel (NL)

(21) Appl. No.: 15/731,672

(22) Filed: Jul. 15, 2017

(51) Int. Cl. A01H 6/48

(52) U.S. Cl. PLT/250

(57) ABSTRACT

A new and distinct cultivar of Hydrangea plant named ‘H216906’, characterized by its upright and rounded plant habit; vigorous growth habit and moderate growth rate; freely branching habit with strong, thick and sturdy stems; early, freely and uniformly flowering habit; mophead-type inflorescences with numerous double-type bright red purple-colored sterile flowers; and good postproduction longevity.

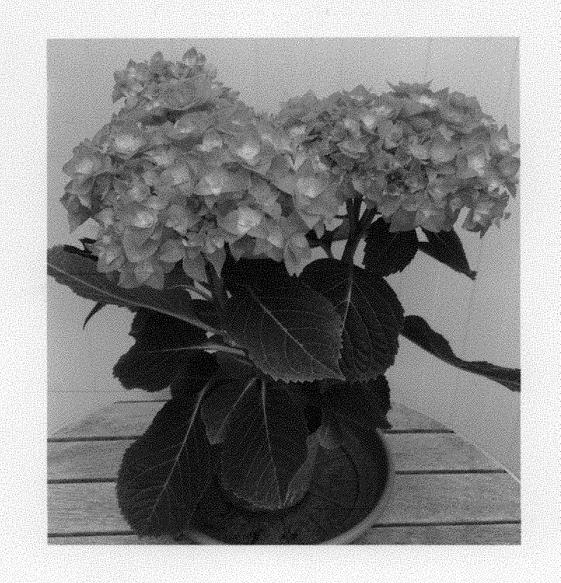

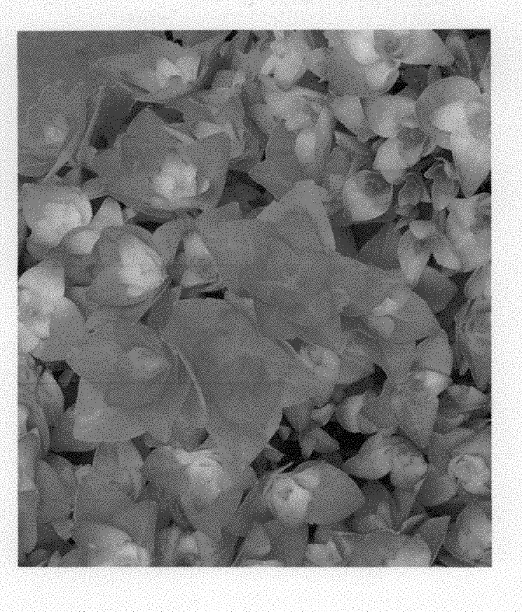

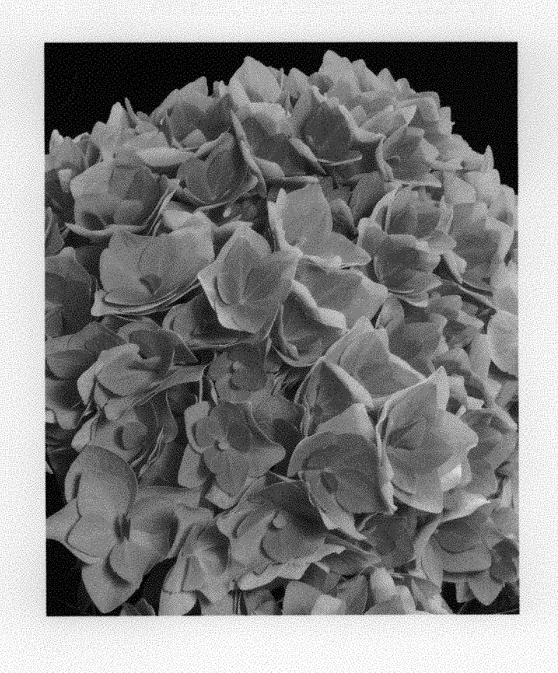

Botanical designation: Hydrangea macrophylla.

Cultivar denomination: ‘H216906’.

### BACKGROUND OF THE INVENTION

The present invention relates to a new and distinct cultivar of Hydrangea plant, botanically known as Hydrangea macrophylla, commercially referred to as a mophead-type Hydrangea and hereinafter referred to by the name ‘H216906’.

The new Hydrangea plant is a product of a planned breeding program conducted by the Inventor in De Kwakel, The Netherlands. The objective of the breeding program was to create new freely-branching Hydrangea plants with strong sturdy stems, large inflorescences with numerous showy sterile flowers, attractive sterile flower color and good postproduction longevity.

The new Hydrangea plant originated from a cross-pollination made by the Inventor in April, 2012 in De Kwakel, The Netherlands, of a proprietary selection of Hydrangea macrophylla identified as code number 10-0169-002, not patented, as the female, or seed, parent with a proprietary selection of Hydrangea macrophylla identified as code number 10-0167-091, not patented, as the male, or pollen, parent. The new Hydrangea plant was discovered and selected by the Inventor as a single flowering plant from within the progeny of the stated cross-pollination in a controlled greenhouse environment in De Kwakel, The Netherlands in March, 2014.

Asexual reproduction of the new Hydrangea plant by vegetative cuttings in a controlled environment in De Kwakel, The Netherlands since June, 2014 has shown that the unique features of this new Hydrangea plant are stable and reproduced true to type in successive generations.

### SUMMARY OF THE INVENTION

Plants of the new Hydrangea have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘H216906’. These characteristics in combination distinguish ‘H216906’ as a new and distinct Hydrangea plant:

1. Upright and rounded plant habit.

2. Vigorous growth habit and moderate growth rate.

3. Freely branching habit with strong, thick and sturdy stems.

4. Early, freely and uniformly flowering habit.

5. Mophead-type inflorescences with numerous double-type bright red purple-colored sterile flowers.

6. Good postproduction longevity.

Plants of the new Hydrangea can be compared to plants of the female and male parent selections. Plants of the new Hydrangea differ primarily from plants of the female and male parent selections in sterile flower type as sterile flowers of plants of the new Hydrangea are double types with several whorls of sepals whereas sterile flowers of plants of the female and male parent selections are single types with a single whorl of sepals.

Plants of the new Hydrangea can be compared to plants of the Hydrangea hybrida ‘Agrihydravijf’, disclosed in U.S. Plant Pat. No. 19,731. In side-by-side comparisons, plants of the new Hydrangea differ primarily from plants of ‘Agrihydravijf’ in the following characteristics:

1. Panicles of plants of the new Hydrangea are broader than panicles of plants of ‘Agrihydravijf’.

2. Plants of the new Hydrangea have more sterile flowers per inflorescence than plants of ‘Agrihydravijf’.

3. Sterile flowers of plants of the new Hydrangea are double types with several whorls of sepals whereas sterile flowers of plants of ‘Agrihydravijf’ are single types with a single whorl of sepals.

4. Plants of the new Hydrangea have darker red purple-colored sterile flower sepals than plants of ‘Agrihydravijf’.

5. When “blued”, plants of the new Hydrangea have purple-colored sterile flower sepals whereas plants of ‘Agrihydravif’ have light violet blue-colored flower sepals.

### BRIEF DESCRIPTION OF THE PHOTOGRAPHS

The accompanying colored photographs illustrate the unique appearance of the new Hydrangea plant showing the colors as true as it is reasonably possible to obtain in colored reproductions of this type. Colors in the photographs may differ from the color values cited in the detailed botanical description which accurately describe the colors of the new Hydrangea plant.

The photograph on the first sheet is a side perspective view of a typical flowering plant of ‘H216906’.

The photograph on the second sheet is a close-up view of a typical inflorescence of that has not been “blued”, that is, treated with aluminum sulfate.

The photograph on the third sheet is a close-up view of a typical inflorescence of ‘H216906’ that has been “blued”.

### DETAILED BOTANICAL DESCRIPTION

Plants used in the aforementioned photographs and in the following description were grown during the spring in 13-cm containers in a glass-covered greenhouse in De Kwakel, The Netherlands and under cultural practices typical of commercial Hydrangea production. During the production of the plants, day and night temperatures averaged 17° C. Plants of the new Hydrangea were pinched one time and were ten months old when the photographs and description were taken. Plants of the new Hydrangea can be successfully treated with aluminum sulfate to “blue” the inflorescences. In the following description, color references are made to The Royal Horticultural Society Colour Chart, 2015 Edition, except where general terms of ordinary dictionary significance are used.
Botanical description: Hydrangea macrophylla ‘H216906’.
Parentage:
   Female, or seed, parent.—Proprietary selection of Hydrangea macrophylla identified as code number 10-0169-002, not patented.
   Male, or pollen, parent.—Proprietary selection of Hydrangea macrophylla identified as code number 10-0167-091, not patented.
Propagation:
   Type cutting.—By vegetative cuttings.
   Time to initiate roots, summer.—About two weeks at temperatures about 23° C.
   Time to initiate roots, winter.—About 18 days at temperatures about 18° C.
   Time to produce a rooted young plant, summer.—About four weeks at temperatures about 23° C.
   Time to produce a rooted young plant, winter.—About five weeks at temperatures about 18° C.
   Root description.—Thick; typically whitish brown in color, actual color of the roots is dependent on substrate composition, water quality, fertilizer type and formulation, substrate temperature and physiological age of roots.
   Rooting habit.—Freely branching; dense.
Plant description:
   Plant and growth habit.—Upright and rounded plant habit; strong and sturdy stems; moderate growth rate and vigorous growth habit.
   Plant height.—About 30 cm to 40 cm.
   Plant diameter or area of spread.—About 30 cm to 40 cm.
Lateral branch description:
   Branching habit.—Freely branching habit; when pinched, about six to eight lateral branches develop per plant.
   Length.—About 25 cm.
   Diameter.—About 4 mm to 5 mm.
   Internode length.—About 3 cm.
   Texture.—Smooth, glabrous; fully developed, woody.
   Aspect.—Upright to about 20° from vertical.
   Strength.—Strong, sturdy.
   Color.—When developing: Close to 144C; at internodes, close to 187B; lenticels, close to 187A. Developed: Close to 144A; at the internodes, close to 187B; when woody, close to 177C; lenticels, close to 187A.
Leaf description:
   Arrangement.—Opposite, simple.
   Length.—About 11 cm.
   Width.—About 6 cm to 8 cm.
   Shape.—Ovate.
   Apex.—Acute.
   Base.—Obtuse.
   Margin.—Serrulate.
   Texture, upper surface.—Smooth to rugose, glabrous.
   Texture, lower surface.—Rugose, glabrous.
   Venation pattern.—Pinnate.
   Color.—Developing and fully developed leaves, upper surface: Close to 147A; venation, close to 145B. Developing and fully developed leaves, lower surface: Close to 137D; venation, close to 145D.
   Petioles.—Length: About 2 cm to 2.5 cm. Diameter: About 3 mm to 4 mm. Texture, upper and lower surfaces: Smooth, glabrous. Color, upper and lower surfaces: Close to 145B.
Flower description:
   Flower type and habit.—Star-shaped double-type sterile flowers arranged on mophead-type terminal panicles; panicles globular in shape; flowers face upright to outwardly depending on their position in the inflorescence; no fertile flower development observed to date.
   Fragrance.—None detected.
   Natural flowering season.—Early flowering habit, plants begin flowering about 41 to 60 weeks after planting; flowering begins in the early summer and is continuous throughout the summer in Northern Europe.
   Flower longevity.—Sterile flowers last about four months on the plant, sterile flowers persistent.
   Quantity of flowers.—Freely flowering habit; about 120 to 160 sterile flowers per panicle.
   Panicle height.—About 10 cm to 14 cm.
   Panicle diameter.—About 15 cm to 20 cm.
   Sterile flower buds.—Length: About 3 mm. Diameter: About 3 mm. Shape: Rounded. Color: Close to 144A.
   Sterile flower diameter.—About 3.5 cm to 4 cm.
   Sterile flower depth (height).—About 5 mm.
   Petals, sterile flowers.—Petal development has not been observed on plants of the new Hydrangea to date.
   Sepals, sterile flowers.—Quantity and arrangement: About 11 to 13 arranged in about three whorls. Length, outer whorl sepals: About 2 cm to 2.5 cm. Length, inner whorl sepals: About 1 cm. Width, outer whorl sepals: About 1.5 cm. Width, inner whorl sepals: About 5 mm. Shape: Rhomboidal. Apex: Acute. Base: Cuneate. Margin: Entire. Texture, upper and lower surfaces: Smooth, glabrous. Color: When opening, upper and lower surfaces: Close to 145C; when “blued”, close to 145C. Fully opened, upper surface: Close to 68A; when “blued”, close to N78B; colors do not change with development. Fully opened, lower surface: Close to 65B; when “blued”, close to N78B; colors do not change with development.
   Pedicels, sterile flowers.—Length: About 2 cm to 3 cm. Diameter: About 2 mm to 3 mm. Strength: Strong. Aspect: Erect to about 45° from vertical. Texture: Smooth, glabrous. Color: Close to 65B.
   Reproductive organs and seeds.—Reproductive organ and/or seed development have not been observed on plants of the new Hydrangea to date.
Disease & pest resistance: Under commercial production conditions, plants of the new Hydrangea have not been observed to be resistant to pathogens and pests common to Hydrangea plants.
Temperature tolerance: Plants of the new Hydrangea have been shown to be tolerant to temperatures ranging from about 3° C. to about 38° C.

It is claimed:

1. A new and distinct Hydrangea plant named ‘H216906’ as illustrated and described.
